(19) United States
(12) Patent Application Publication
(10) Pub. No.: US 2007/0029521 A1
(43) Pub. Date: Feb. 8, 2007

(54) High speed liquid crystalline compounds

(75) Inventors: Pao Ju Hsieh, Hsinchu City (TW); Hui Lung Kuo, Taipei City (TW); Pin Chen Chen, Taipei City (TW); Yi Ping Hsieh, Changhua County (TW); Shin Tson Wu, Nantou County (TW); Sebastian Gauza, Orlando, CA (US)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsin Chu (TW)

(21) Appl. No.: 11/299,781

(22) Filed: Dec. 13, 2005

(30) Foreign Application Priority Data
Aug. 4, 2005 (TW) 94126513

(51) Int. Cl. C09K 19/30; C09K 19/12; C07C 25/13

(52) U.S. Cl. 252/299.63; 252/299.66; 570/127

(57) ABSTRACT

The present invention provides a novel high speed liquid crystalline compound with high birefringence and low rotational viscosity, comprising a general formula as formula (1): 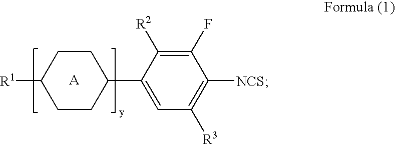
wherein R¹ comprises C₁-C₁₂ alkyl or alkoxy groups, R², R³ independently represents H or F, y represents an integer of 1 or 2, and A is selected from 1,4-phenylene or 1,4-trans-cyclohexylene.

### FIELD OF THE INVENTION

The present invention discloses a novel liquid crystalline compound having high speed, high birefringence, and low viscosity.

### BACKGROUND OF THE INVENTION

Since RCA first introduced liquid crystal display in 1968 utilizing the phase transfer property and low driving voltage phenomenon of liquid crystal, liquid crystalline compounds have been applied to the manufacturing of flat panel display. In the age of multimedia, the demands of flat panel display are on the rise. In addition, as liquid crystal display offers the advantages of light weight, thin, short, small-size, saving energy and color display, it is also extensively applied in electronic devices, such as calculator, dashboard, portable liquid crystal TV, laptop, large-screen high definition TV (HDTV), personal digital assistant (PDA), and global positioning system in cars. Governments around the world have put in considerable efforts to develop their liquid crystal industry.

Liquid crystal display utilizes field effect to alter the alignment of liquid crystal molecules, which leads to the polarization and direction change of light and produces the contrast effect. But the response speed of liquid crystal display is slower than other self-luminous display devices, often resulting in residual images on the screen. This is also why liquid crystal displays in the early days were not suitable for animated display. In recent years with the fabrication of liquid crystal cells and the aid of driver circuit technology, liquid crystal display has made significant improvement in terms of response speed. But the cases where simply utilizing the properties of liquid crystal material to improve response speed are rather few. Thus developing new liquid crystalline compounds to improve response speed has become one of the most important topics in related fields.

### SUMMARY OF THE INVENTION

In light of the need to improve the response speed of liquid crystal and the drawbacks of prior art, the present invention aims to provide a novel high speed liquid crystalline compound. From the response speed formula

$${τ}_{o} ≅ \frac{{y}_{1}}{{K}_{11}} {( \frac{d}{π} )}^{2} ,$$

it is known that liquid crystal response speed is correlated with rotational viscosity (γ₁). To increase the response speed, the primary condition is to reduce the viscosity of liquid crystal, in particular rotational viscosity (γ₁). Also under the consideration of reducing the thickness of liquid crystal cell to speed up the response, the birefringence of liquid crystal molecules must be enhanced to maintain certain contrast ratio. Therefore the object of the present invention is to provide a novel liquid crystalline compound with high response speed, high birefringence and low rotational viscosity, comprising a general formula as following formula (1): 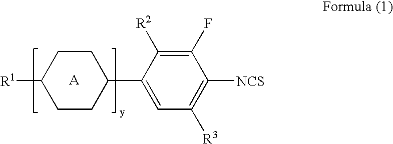
wherein R¹ comprises C₁-C₁₂ alkyl or alkoxy groups, R², R³ independently represents H or F, y represents an integer of 1 or 2, and A is selected from 1,4-phenylene or 1,4-trans-cyclohexylene.

When R² and R³ are independently H, an embodiment of the aforesaid liquid crystalline compound comprises a molecular structure as shown in formula (2): 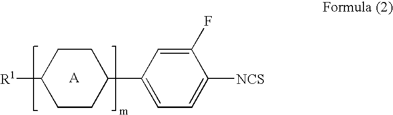
wherein R¹ and A are defined as the same above, and m is 1 or 2.

When R³ is F and R² is H, an embodiment of the aforesaid liquid crystalline compound comprises a molecular structure as shown in formula (3): 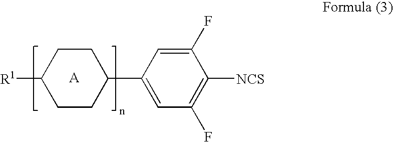
wherein R¹ and A are defined the as same above, and n is 1 or 2.

When R² and R³ in the liquid crystalline compound of formula (1) are independently F and H, an embodiment of the aforesaid liquid crystalline compound comprises a molecular structure as shown in formula (4): 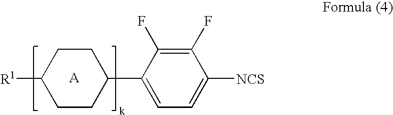
wherein R¹ and A are defined as the same above, and k is 1 or 2.

When m=1, and A is 1,4-phenylene in the liquid crystalline compound of formula (2), liquid crystalline compound comprises a molecular structure as shown in formula (2-1): 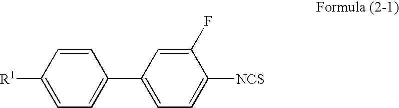
wherein R¹ is defined as the same above.

The liquid crystalline compound of formula (2-1) has an embodiment, comprising a molecular structure as shown in formula (2-1-a):

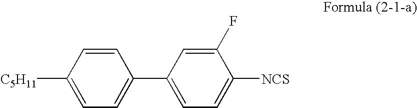

For the liquid crystalline compound as shown in formula (2), when m=2, and A is 1,4-phenylene and 1,4-trans-cyclohexylene, it comprises compounds having the structures as shown in formulas (2-2), (2-3), and (2-4): 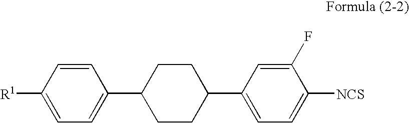 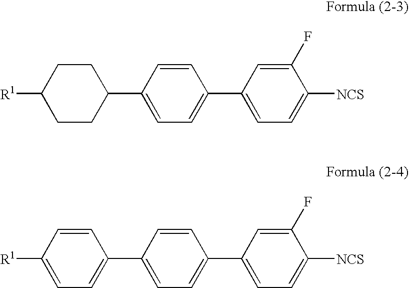
wherein R¹ is defined as the same above.

For the liquid crystalline compound as shown in formula (3), when n=1, and A is 1,4-phenylene or 1,4-trans-cyclohexylene, it comprises compounds having the structures as shown in formulas (3-1) and (3-2): 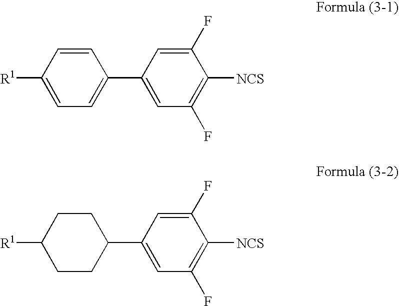
wherein R¹ is defined as the same above.

In addition, for the liquid crystalline compound as shown in formula (3), when n=2, and A is 1,4-phenylene and 1,4-trans-cyclohexylene, it comprises a compound having a structure as shown in formula (3-3): 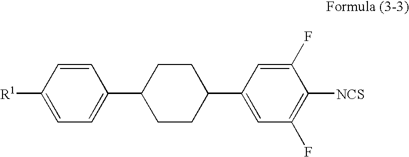
wherein R¹ is defined as the same above.

For the liquid crystalline compound as shown in formula (4), when k=l, and A is 1,4-phenylene or 1,4-trans-cyclohexylene, it comprises compounds having the structures as shown in formulas (4-1) and (4-2): 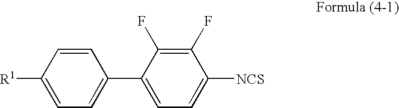 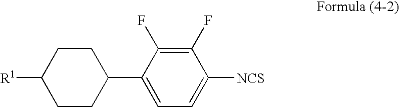
wherein R¹ is defined as the same above.

For the liquid crystalline compound as shown in formula (4), when k=2, and A is 1,4-phenylene or 1,4-trans-cyclohexylene, it comprises compounds having the structures as shown in formulas (4-3) to (4-6): 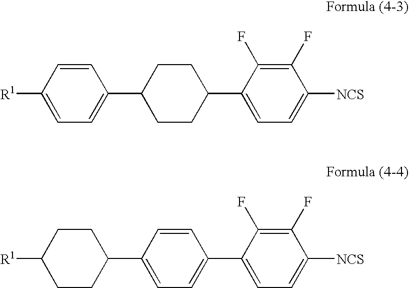 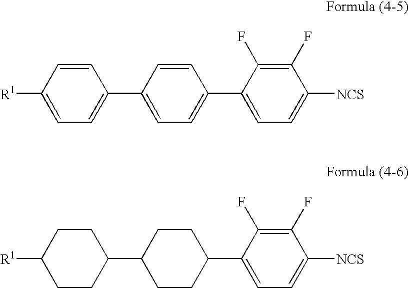
wherein R¹ is defined as the same above.

The present invention further provides a high speed liquid crystal mixture, comprising any of the compounds of formula (1) or mixture thereof.

The high speed liquid crystalline compound of the present invention is characterized by simultaneously having NCS and fluorine functional group on its terminal ring, wherein the former provides strong dipolemoment which effectively enhances the birefringence of liquid crystal molecules, while the incorporated fluorine group on the side slightly reduces the van der Waals force between molecules to decrease the viscosity of liquid crystal, and the second and the third rings thereafter provide the foundation for the formation and maintenance of liquid crystal phase. The design theory of the present invention of adjusting birefringence is entirely different from the majority of prior arts that use phenylcyclohexane with smaller viscosity as core structure and contain three or more rngs.

### DETAILED DESCRIPTION OF THE INVENTION

### EXAMPLE 1

### The Steps for Synthesizing Liquid Crystalline Compounds (7), (8) of the Present Invention Are as Follows:

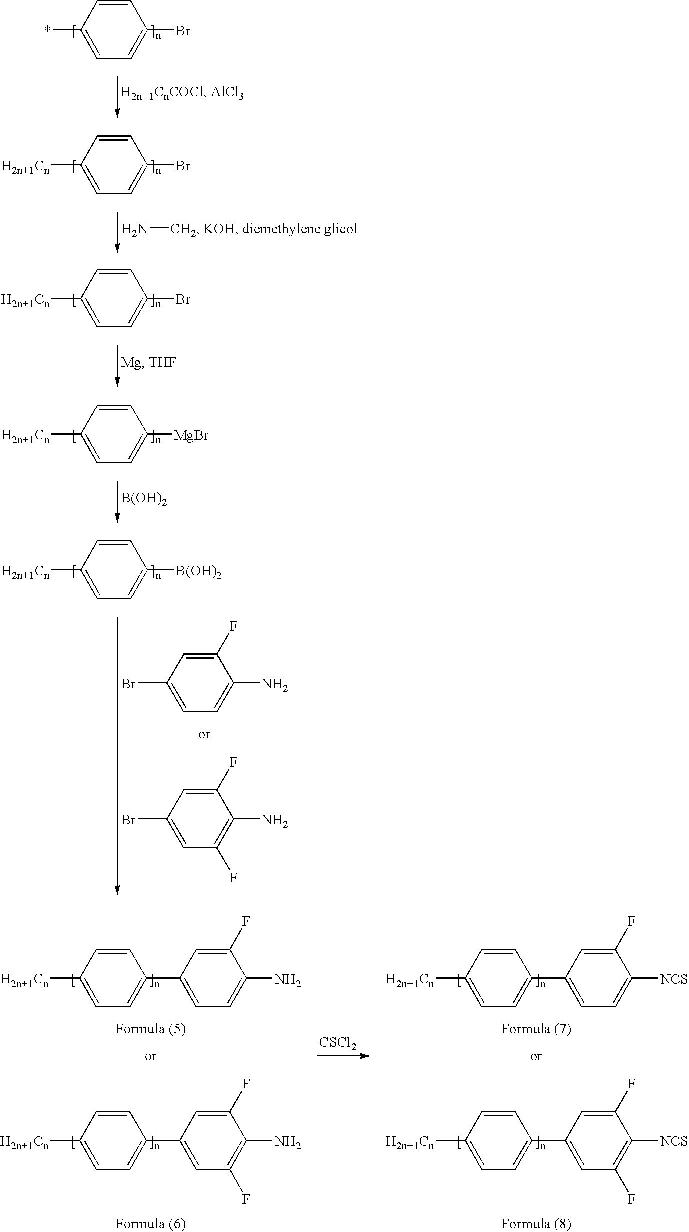 wherein n is 1 or 2.

Formula (7) and formula (8) are respectively embodiments of formula (2) and formula (3), when m=1, R¹ is alkyl, and A is 1,4-phenylene.

Additionally, in the synthesis process, the present invention would produce two intermediates having the structures as shown in formula (5) and formula (6) respectively. As shown, the terminal ring of the two intermediates carries fluorine function group which slightly reduces the van der Waals force between molecules to decrease the viscosity of liquid crystal, thereby achieving the objective of increasing response speed. Moreover, the high speed liquid crystalline compound in formula (7) or formula (8), produced according to the synthesis steps above, can also be obtained by replacing the —NH₂ group on the terminal ring of intermediate of formula (5) or formula (6) with —NCS group through other known chemical reactions in the art to provide strong dipolemoment, thereby effectively enhancing the birefringence of liquid crystal molecules. The process for synthesizing the high speed liquid crystalline compound based on the present invention is not limited to the steps described above. Any person skilled in the art is able to make any modification to the aforesaid method to prepare the compound disclosed in the present invention.

### EXAMPLE 2

### Comparing the Photoelectric Properties of Liquid Crystalline Compounds of the Present Invention with Those in the Prior Art

To demonstrate the superior photoelectric properties of the liquid crystalline compounds disclosed in the present invention, compound of formula (2-1-a) with birefringence of 0.23 is used for testing, in which MLC 13900-100 (Merck Co.) is used as mother solution and comparison. The percentages of compound of formula (2-1-a) and supplements are shown in Table 1. According to the measurements of photoelectric properties as depicted in Table 2, it is apparent that the composition added with the liquid crystalline compound of the present invention shows marked improvement in birefringence, dielectric constant, driving voltage, rotational viscosity, and response time. Thus the design of the present invention offers great potential for the development of high speed liquid crystal.

TABLE 1

| | |
|---|---|
| | Formula (2-1-a) |
| 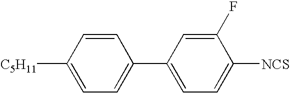 | |

| | Composition according to | |
|---|---|---|
| | the present invention | Comparison |
| Code | (MRL-HS1) | (MLC 13900-100) |
| MLC 13900-100 | 66.7 wt % | 100 wt % |
| Compound of | 33.3 wt % | |
| formula (2-1-a) | | |

TABLE 2

| | | Composition according | |
|---|---|---|---|
| | | to the present | Comparison |
| | Code | invention (MRL-HS1) | (MLC 13900-100) |
| Birefringence | Δn | 0.1561 | 0.1058 |
| Driving voltage | Vₜₕ | 1.74 [V] | 2.3 [V] |
| Dielectric | ε_{II} | 11.0 | 8.0 |
| constant | ε⊥ | 3.7 | 3.1 |
| | Δε | 7.3 | 4.9 |
| Elasticity | κ₁₁ | 1.15e⁻¹¹[N] | 1.33e⁻¹¹[N] |
| coefficient | κ₃₃ | 3.51e⁻¹¹[N] | 3.88e⁻¹¹[N] |
| Rotational | γ₁ | 90 | 116 |
| viscosity | γ₁/κ₁₁ | 7.8 | 8.6 |
| Response time | τ | 7.307 [um/s] | 13.94 [um/s] |
| | | (d = 3 μm) | (d = 4 μm) |

In summary, the present invention provides a novel high speed liquid crystalline compound, which as comparing to prior art, has shorter response time, high birefringence and low rotational viscosity, hence possessing great commercial value for application in the manufacture of liquid crystal display.

What is claimed is:

1. A high speed liquid crystalline compound comprising the structure as shown in formula (1): 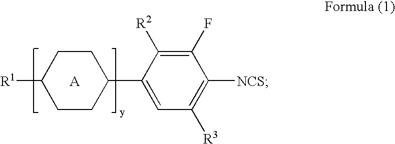
wherein R¹ comprises C₁-C₁₂ alkyl or alkoxy groups, R , R independently represents hydrogen or fluorine, y represents an integer of 1 or 2, and A is selected from 1,4-phenylene or 1,4-trans-cyclohexylene.

2. The high speed liquid crystalline compound according to claim 1, wherein when R² and R³ are independently hydrogen, said liquid crystalline compound comprises a structure as shown in formula (2): 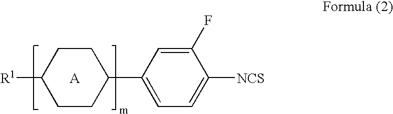
wherein R¹ and A are defined as the same above, and m is an integer of 1 or 2.

3. The high speed liquid crystalline compound according to claim 1, wherein when R³ is fluorine and R² is hydrogen, said liquid crystalline compound comprises a structure as shown in formula (3): 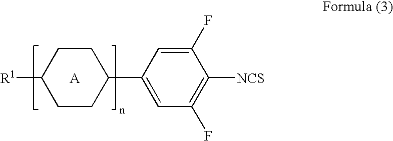
wherein R¹ and A are defined as the same above, and n is an integer of 1 or 2.

4. The high speed liquid crystalline compound according to claim 1, wherein when R² is fluorine and R³ is hydrogen, said liquid crystalline compound comprises a structure as shown in formula (4): 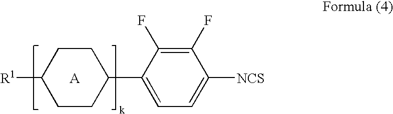
wherein R¹ and A are defined as the same above, and k is an integer of 1 or 2.

5. The high speed liquid crystalline compound according to claim 2, wherein when m=1, and A is 1,4-phenylene, said liquid crystalline compound comprises a molecular structure as shown in formula (2-1): 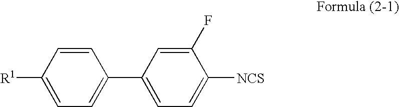
wherein R¹ is defined the same as above.

6. The high speed liquid crystalline compound according to claim 5, comprising a compound having a structure as shown in formula (2-1-a):

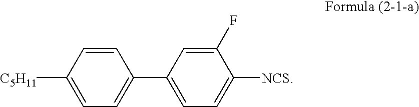

7. The high speed liquid crystalline compound according to claim 2, comprising compounds having the structures as shown in formulas (2-2), (2-3), and (2-4), when m=2, and A is 1,4-phenylene and 1,4-trans-cyclohexylene: 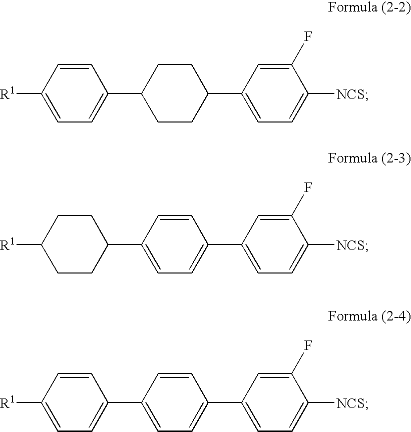
wherein R¹ is defined as the same above.

8. The high speed liquid crystalline compound according to claim 3, comprising compounds having the structures as shown in formulas (3-1) and (3-2), when n=1, and A is 1,4-phenylene or 1,4-trans-cyclohexylene: 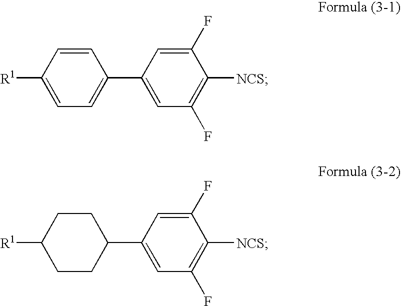
wherein R¹ is defined as the same above.

9. The high speed liquid crystalline compound according to claim 3, comprising a compound having the structure as shown in formula (3-3), when n=2, and A is 1,4-phenylene and 1,4-trans-cyclohexylene: 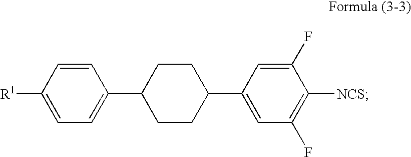
wherein R¹ is defined as the same above.

10. The high speed liquid crystalline compound according to claim 4, comprising compounds having the structures as shown in formulas (4-1) and (4-2), when k=1, and A is 1,4-phenylene or 1,4-trans-cyclohexylene: 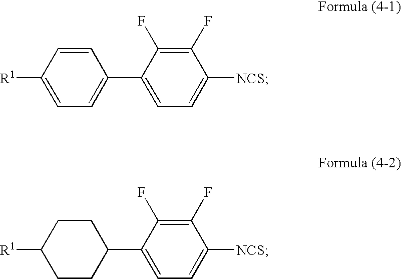
wherein R¹ is defined as the same above.

11. The high speed liquid crystalline compound according to claim 4, comprising compounds having the structures as shown in formulas (4-3) to (4-6), when k=2, and A is 1,4-phenylene or 1,4-trans-cyclohexylene: 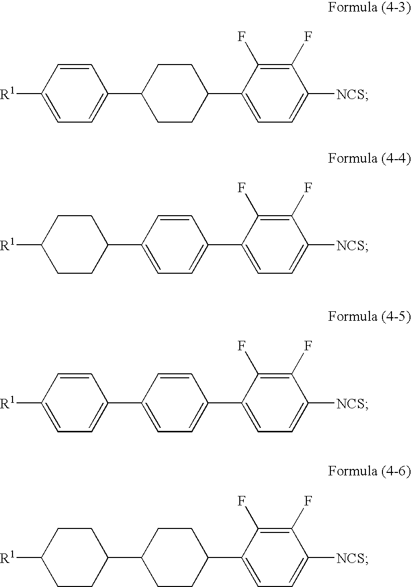
wherein R¹ is defined as the same above.

12. The high speed liquid crystalline compound according to claim 2, wherein when m=1, R¹ is alkyl, and A is 1,4-phenylene in the formula (2), said liquid crystalline compound comprises a structure as shown in formula (7): 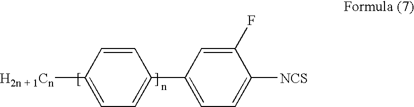
wherein n is an integer of 1 or 2.

13. The high speed liquid crystalline compound according to claim 3, wherein when n=1, R¹ is alkyl, and A is 1,4-phenylene in the formula (3), said liquid crystalline compound comprises a structure as shown in formula (8): 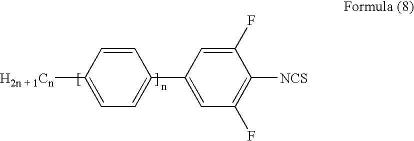
wherein n is an integer of 1 or 2.

14. A high speed liquid crystal mixture, comprising a compound of formula (1) according to claim 1 or mixture thereof.
